(19) United States
(12) Patent Application Publication
(10) Pub. No.: US 2026/0083821 A1
(43) Pub. Date: Mar. 26, 2026

(54) Compositions and Methods for Treating Cardiovascular Diseases and Disorders

(71) Applicant: Olfactive AI, Inc., Mountain View, CA (US)

(72) Inventor: Christopher Hanson, Mountain View, CA (US)

(21) Appl. No.: 19/403,633

(22) Filed: Nov. 28, 2025

Related U.S. Application Data
(63) Continuation-in-part of application No. 19/070,810, filed on Mar. 5, 2025.
Application No. 19/070,810 is a continuation-in-part of application No. 18/980,129, filed on Dec. 13, 2024, now Pat. No. 12,290,550.
Application No. 18/980,129 is a continuation-in-part of application No. 18/892,760, filed on Sep. 23, 2024.
Application No. 18/892,760 is a division of application No. 18/615,150, filed on Mar. 25, 2024, now Pat. No. 12,102,664.
Application No. 18/615,150 is a continuation-in-part of application No. 18/430,796, filed on Feb. 2, 2024, now Pat. No. 12,115,134.
Application No. 19/070,810 is a continuation-in-part of application No. 18/958,791, filed on Nov. 25, 2024.
Application No. 18/958,791 is a continuation of application No. 18/811,171, filed on Aug. 21, 2024, now Pat. No. 12,186,299.
Application No. 18/811,171 is a division of application No. 18/615,452, filed on Mar. 25, 2024, now Pat. No. 12,102,611.
(60) Provisional application No. 63/615,100, filed on Dec. 27, 2023.

(51) Int. Cl. A61K 38/26; A61K 31/085; A61K 31/202

(52) U.S. Cl. CPC A61K 38/26; A61K 31/085; A61K 31/202

(57) ABSTRACT

Disclosed herein are compounds and ligands, and compositions formed therewith, that treat insulin resistance-induced physiological disorders by modulating G-protein coupled receptor (GPCR) and transient receptor potential (TRP) ion channel activity. Also disclosed herein are methods for using the compositions to treat cardiovascular disorders, such as atherosclerosis.

### CROSS-REFERENCES TO RELATED APPLICATIONS

This application is a continuation-in-part of U.S. application Ser. No. 19/070,810, filed on Mar. 5, 2025, which is a continuation-in-part of U.S. application Ser. No. 18/980,129, filed on Dec. 13, 2024, now U.S. Pat. No. 12,290,550, which is a continuation-in-part of U.S. application Ser. No. 18/892,760, filed on Sep. 23, 2024, which is a divisional of U.S. application Ser. No. 18/615,150, filed on Mar. 25, 2024, now U.S. Pat. No. 12,102,664, which is a continuation-in-part of U.S. application Ser. No. 18/430,796, filed on Feb. 2, 2024, now U.S. U.S. Pat. No. 12,115,134, which claims the benefit of U.S. Provisional Application No. 63/615,100, filed on Dec. 27, 2023.

Parent U.S. application Ser. No. 19/070,810 noted above is also a continuation-in-part of U.S. application Ser. No. 18/958,791, filed on Nov. 25, 2024, which is a continuation application of U.S. application Ser. No. 18/811,171, filed on Aug. 21, 2024, now U.S. Pat. No. 12,186,299, which is a divisional application of U.S. application Ser. No. 18/615,452, filed on Mar. 25, 2024, now U.S. Pat. No. 12,102,611.

### FIELD OF THE INVENTION

The present invention relates to compositions and methods for treating insulin resistance-induced physiological disorders. More particularly, the present invention relates to compositions and methods for treating cardiovascular disorders by modulating receptor activity.

### BACKGROUND OF THE INVENTION

As is well established, insulin is a potent anabolic hormone that exerts a variety of effects on many types of cells. Some of the main metabolic actions of insulin are stimulating glucose uptake in skeletal muscles and adipocytes, promoting glycogen synthesis in skeletal muscles, suppressing hepatic glucose production, and inhibiting lipolysis in adipocytes.

In most instances, insulin secretion is induced by pancreatic β-cells when glucagon-like peptide-1 (GLP-1) binds to and activates GLP-1 receptor proteins on endogenous gastrointestinal (GI) cells, such as enteroendocrine L-cells.

As is also well established, in metabolically impaired subjects, abnormal insulin secretion and loss of cellular sensitivity to insulin signaling (insulin resistance) can occur, which principally affects liver, muscle, and adipose cells and is selective for glucose and lipid metabolism. Insulin resistance results in a reduction in insulin-mediated glucose uptake by endogenous cells and further results in compensatory hypersecretion of insulin by pancreatic β-cells to maintain homeostasis. The hypersecretion of insulin by β-cells typically leads to β-cell exhaustion and dysfunction, and ultimately impairment of insulin production by the β-cells (i.e., abnormal insulin secretion).

As is additionally well established, abnormal insulin secretion and associated insulin resistance are associated with various insulin resistance-induced physiological disorders; particularly, type 2 diabetes mellitus. The prevalence of type 2 diabetes mellitus continues to rise worldwide as lifestyles associated with low energy expenditure and high caloric intake and, hence, metabolic dysfunction, are increasingly adopted, particularly in lower-income and developing countries. Indeed, it is estimated that the number of cases of type 2 diabetes mellitus is projected to rise from 830 million worldwide to 1.3 billion by 2050.

Recent studies also reflect that there is also a strong correlation between insulin resistance and, hence, hyperglycemia associated therewith, and various cardiovascular disorders, such as atherosclerosis by virtue of vascular endothelial dysfunction, vascular smooth muscle cell (VSMC) over-proliferation and migration, and monocyte/macrophage/foam cell accumulation associated therewith.

Recent studies thus also reflect that there is a strong correlation between abnormal insulin secretion and various cardiovascular disorders, such as atherosclerosis.

Various entities have thus developed pharmaceutically active agents and compositions that are adapted to treat abnormal insulin secretion. In view of the strong correlation between activation of the GLP-1 receptor proteins and insulin secretion, the pharmaceutically active agents and compositions are specifically adapted to activate the GLP-1receptor proteins on endogenous gastrointestinal (GI) cells.

Such pharmaceutically active agents include semaglutide (Ozempic®, Rybelsus®, Wegovy®), dulaglutide (Trulicity®), exenatide (Bydureon BCise®, Byetta®), and liraglutide (Victoza®, Saxenda®).

The noted pharmaceutically active agents (referred to hereinafter as “GLP-1 analogs”) mimic endogenous GLP-1 and are adapted to activate the GLP-1 receptors on endogenous GI cells and, hence, function as GLP-1 receptor agonists.

Although the GLP-1 analogs can effectively activate GLP-1 receptors on pancreatic β-cells and, hence, can induce insulin secretion and, thereby, effectively treat various physiological disorders, there are several drawbacks and disadvantages associated with administration of the GLP-1 analogs to subjects.

A major drawback associated with the administration of the GLP-1 analogs to subjects is the high risk of adverse pathological events. One such adverse pathological event is hypoglycemia (i.e., low blood glucose), which can, and often will, be present in subjects that are also taking or being administered commonly prescribed antidiabetic agents, such as basal insulin and sulfonylureas.

There is also a high risk of induced production of anti-GLP-1 antibodies and binding of endogenous GLP-1 and the GLP-1 analogs to the anti-GLP-1 antibodies, which can, and often will, induce adverse immune responses.

A further major drawback associated with the administration of GLP-1 analogs to subjects are the significant side effects that are often presented by the subjects, including nausea, vomiting, diarrhea, abdominal pain, and constipation.

Since most GLP-1 analogs are administered to subjects via a subcutaneous injection, a further drawback associated with GLP-1 analog administration is the pain and discomfort associated with the often-prescribed weekly injections.

Although the GLP-1 analogs developed by Novo Nordisk, which are marketed under the tradename Rybelsus, can also be delivered orally, a significantly greater dose of the Rybelsus GLP-1 analog must be orally administered to an subject to match the pharmacokinetics of the Novo Nordisk injectable GLP-1 analog, which is marketed under the tradename Ozempic, i.e., subjects must be orally administered approximately 100.0 mg/week of the Rybelsus GLP-1 analog to match the efficacy of the typically prescribed 0.5 mg/week of the injectable Ozempic GLP-1 analog.

A further major drawback associated with the administration of GLP-1 analogs to subjects is the cost. Indeed, the costs, at present, for a thirty (30) day supply of Ozempic and Rybelsus are approximately $1000.00 and $1200.00, respectively.

As is also well established, insulin secretion can also be induced by pancreatic β-cells when gastric inhibitory polypeptide (GIP) binds to and activates GIP receptor proteins on the pancreatic β-cells.

Although GIP also induces insulin secretion, there are similarly several drawbacks and disadvantages associated with solely activating the GIP receptor proteins on the pancreatic β-cells.

A major disadvantage associated with solely activating the GIP receptor proteins on the pancreatic β-cells is that GIP also induces glucagon secretion from pancreatic β-cells. Since glucagon is a hyperglycemic compound that increases blood sugar when secreted, the increase in glucagon secretion induced by GIP limits its therapeutic potential for treating abnormal insulin secretion.

To address the above noted disadvantage associated with solely activating the GIP receptor proteins on the pancreatic β-cells, Eli Lilly has recently developed a dual GLP-1/GIP analog that activates GLP-1 and GIP receptors on pancreatic β-cells.

The dual GLP-1/GIP analog, i.e., tirzepatide, which is marketed under the tradenames Mounjaro® and ZepBound®, provides the beneficial metabolic activity induced by both GLP-1 and GIP in a synergistic manner without a clinically significant increase in glucagon secretion.

Many of the drawbacks and disadvantages associated with administration of the GLP-1 analogs to subjects are, however, also associated with administration of the dual GLP-1/GIP analog to subjects.

Such drawbacks and disadvantages include the significant side effects that are often presented by the subjects, including nausea, vomiting, diarrhea, abdominal pain, kidney problems and constipation, and high cost.

There is thus a need for compounds and ligands; particularly, natural compounds and ligands, and compositions comprising same, for treating physiological disorders; particularly, insulin resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to subjects presenting with a physiological disorder.

It is thus one object of the present invention to provide natural compounds and ligands, and compositions comprising same, for treating physiological disorders; particularly, insulin resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to subjects presenting with a physiological disorder.

It is another object of the present invention to provide natural compounds and ligands, and compositions comprising same, which, when administered to subjects presenting with a physiological disorder, effectively treat the physiological disorder by modulating ectopic olfactory receptor activity.

It is another object of the present invention to provide natural compounds and ligands, and compositions comprising same, which effectuate olfactory receptor (OR)-mediated secretion of endogenous GLP-1 and PYY in subjects presenting with a physiological disorder, without the undesirable side effects associated with delivery of a GLP-1 analog and/or a dual GLP-1/GIP analog to subjects.

It is another object of the present invention to provide natural compounds and ligands, and compositions comprising same, which, when administered to subjects presenting with a cardiovascular disorder, effectively and safely modulate the subjects'insulin secretion in vivo, whereby the cardiovascular disorder is effectively treated and at least one risk factor associated with the cardiovascular disorder is ameliorated with minimal, if any, adverse side effects.

It is another object of the present invention to provide natural compounds and ligands, and compositions comprising same, which, when administered to subjects presenting with a cardiovascular disorder, effectively and safely modulate the subjects'insulin secretion and abates 5-HT₂-serotonin receptor activity in vivo, whereby the cardiovascular disorder is effectively treated and at least one risk factor associated with the cardiovascular disorder is ameliorated with minimal, if any, adverse side effects.

It is another object of the present invention to provide natural compounds and ligands, and compositions comprising same, which, when administered to subjects, effectively and safely modulate the subjects'systemic insulin secretion and abates 5-HT₂-serotonin receptor activity in vivo, and can be administered to the subjects via oral, sublingual, inhalation, intranasal, epidural, intracerebral, transdermal, topical, and injection administration means.

### SUMMARY OF THE INVENTION

The present invention is directed to compositions and methods for treating insulin-resistance-induced physiological disorders; particularly, cardiovascular disorders.

In some embodiments of the invention, there are thus provided methods for treating atherosclerosis presented by a subject.

In one embodiment of the invention, the method for treating atherosclerosis presented by a subject comprises the following steps:
providing a composition comprising a delivery medium, at least a first receptor activating compound, at least a second receptor activating compound and a receptor antagonizing compound,
the first receptor activating compound comprising butyl butyryl lactate, the butyl butyryl lactate comprising in the range of approximately 3.0% to approximately 5.0% (w/w) of the composition,
the second receptor activating compound comprising lauric acid, the lauric acid comprising in the range of approximately 0.1% to approximately 0.5% (w/w) of the composition,
the receptor antagonizing compound comprising 3,3′,4′,5,6,7,8-heptamethoxyflavone (HMF), the HMF comprising in the range of approximately 15.0% to approximately 17.0% (w/w) of the composition,
the composition adapted to induce activation of at least olfactory receptor family 51 subfamily E member 1 (OR51E1) and free fatty acid receptor 1 (FFAR1) activity in vivo, and induce abatement of 5-HT₂-serotonin receptor activity in vivo; and
delivering the composition to the subject, wherein secretion of glucagon-like peptide-1 (GLP-1) and gastric inhibitory polypeptide (GIP) is induced, and atherosclerotic platelet aggregation is abated.

In another embodiment of the invention, the method for treating atherosclerosis presented by a subject comprises the following steps:
providing a composition comprising a delivery medium, at least a first receptor activating compound, at least a second receptor activating compound, at least a third receptor activating compound, and a receptor antagonizing compound,
the first receptor activating compound comprising butyl butyryl lactate, the butyl butyryl lactate comprising in the range of approximately 3.0% to approximately 5.0% (w/w) of the composition,
the second receptor activating compound comprising lauric acid, the lauric acid comprising in the range of approximately 0.1% to approximately 0.5% (w/w) of the composition,
the third receptor activating compound comprising cinnamaldehyde, the cinnamaldehyde comprising in the range of approximately 24.0% to approximately 26.0% (w/w) of the composition,
the receptor antagonizing compound comprising 3,3′,4′,5,6,7,8-heptamethoxyflavone (HMF), the HMF comprising in the range of approximately 15.0% to approximately 17.0% (w/w) of the composition,
the composition adapted to induce activation of at least olfactory receptor family 51 subfamily E member 1 (OR51E1) and free fatty acid receptor 1 (FFAR1) activity in vivo, and induce abatement of 5-HT₂-serotonin receptor activity in vivo; and
delivering the composition to the subject, wherein secretion of glucagon-like peptide-1 (GLP-1) and gastric inhibitory polypeptide (GIP) is induced, and atherosclerotic platelet aggregation is abated.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A, 1B, and 1C are schematic illustrations of 5-HT₂ receptor-mediated atheroma formation in an arterial blood vessel;

FIG. 2 is a schematic illustration of receptor-mediated activation of GLP-1 secretion from endogenous gastrointestinal cells;

FIG. 3 is a schematic illustration of receptor-mediated activation of GIP secretion from endogenous gastrointestinal cells;

FIG. 4 is a bar graph depicting induced OR51E1 activity, expressed as luminescence emanating from cells in vitro, by butyl butyryl lactate at various concentrations;

FIG. 5 is a bar graph depicting induced FFAR1 activity, expressed as luminescence emanating from cells in vitro, by lauric acid at various concentrations; and

FIG. 6 is a bar graph depicting induced FFAR1 activity, expressed as luminescence emanating from cells in vitro, by butyl butyryl lactate, lauric acid, and a mixture of butyl butyryl lactate and lauric acid at concentrations of 1500.0 μM.

### DETAILED DESCRIPTION OF THE PREFERRED EMBODIMENT

Before describing the present invention in detail, it is to be understood that this invention is not limited to particularly exemplified compounds, compositions or methods, as such may, of course, vary. Thus, although a number of compounds, compositions and methods similar or equivalent to those described herein can be used in the practice of the present invention, the preferred compounds, compositions and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Thus, for example, reference to “an active agent”includes two or more such agents and the like.

### Definitions

The terms “cardiovascular disorder” and “cardiovascular diseases”, as used herein, are used interchangeably herein and mean and include any physiological disorder associated with abnormal heart and blood vessel/circulatory system function, such as atherosclerosis.

The term “olfactory receptor (OR)” as used herein, means and includes an olfactory receptor that is a seminal component of the chemosensory organs responsible for olfaction. The term “olfactory receptor” as used herein, also means, and includes, trace amine associated receptors, vomeronasal receptors, formyl peptide receptors, membrane guanylyl cyclase, subtype GC-D receptors; and G-protein coupled receptors, such as G-protein coupled taste receptors. Olfactory receptors can also include hybrid receptors synthesized from the above-noted olfactory receptors.

The term “ectopic olfactory receptor”, as used herein, means and includes an olfactory receptor that is present in organs, tissue, and/or cells that is a seminal component of physiological processes outside of olfaction and, in some instances, indirectly involved with olfactory-mediated processes.

The terms “olfaction” and “olfactory reception” are used interchangeably herein, mean and include the interaction of a composition (or formulation) with an olfactory receptor coupled to a cell signaling pathway. The composition can also be defined as an “odorant” and may be airborne (i.e., volatile) and/or in solution.

The term “free fatty acid receptor”, as used herein, means and includes a transmembrane cell surface receptor that is adapted and configured to bind to fatty acids and induce cell signaling processes in response to the binding of the fatty acids.

The term “transient receptor potential ion channel”, as used herein, means and includes a transmembrane ion channel that is adapted and configured to modulate ion entry into an endogenous cell, such as Ca²⁺ entry, and, thereby, induce cell signaling process when a compound or ligand binds to the ion channel.

The term “insulin resistance”, as used herein, means, and includes a condition in which insulin exerts a biological effect that is lower than expected, due to defects in insulin-stimulated glucose uptake; particularly, in glycogen synthesis and, to a lesser extent, glucose oxidation.

The term “insulin-resistance-induced physiological disorder”, as used herein, thus means and includes a physiological disease and/or a physiological disorder characterized by metabolic dysfunction and conditions associated therewith including, but not limited to, dysfunction of glucose metabolism and attendant insulin resistance.

The term “modulation,” as used herein in connection with insulin, means and includes, without limitation, activating and/or regulating at least one cellular process relating to production, synthesis and transmission of insulin, including, but not limited to (i) production and, hence, release of the insulin via activation of a receptor, e.g., GLP-1 receptor and GIP receptor activation, (ii) binding of the released insulin to insulin receptors, (iii) synthesis of the insulin, (iv) alteration(s) of intracellular signaling pathways associated with insulin synthesis and secretion, and (v) alteration(s) of cellular sensitivity to extracellular insulin.

The term “agonist” as used herein, means, and includes any molecule which binds to a receptor on a cell, wherein the binding to the receptor can potentially lead to subsequent changes in the cell's functions. When an agonist binds to a sufficient number of receptors, the receptors can activate seminal processes in the cell.

The term “antagonist”, as used herein, means and includes a molecule and/or a compound comprising same, which binds to a receptor on a cell and inhibits the receptor from activating processes in the cell. The inhibition of the receptor can include competitive binding against agonists (when an antagonist is bound, agonists cannot bind to the receptor) and allosteric effects (when the antagonist binds, agonists can still bind the receptor, but cannot activate the receptor).

The term “receptor antagonizing compound”, as used herein, thus means and includes an “antagonist.”

The term “endocrine factor” as used herein, means and includes any molecule and/or a compound comprising same, which is produced and secreted by endogenous cells and induces biological activity at a biological tissue site. The term “endocrine factor” thus means and includes, without limitation, insulin, glucagon-like peptide-1 (GLP-1), gastric inhibitory polypeptide (GIP), peptide Y-Y (PYY), ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, glucagon, enteroglucagon, galanin, leptin, motilin, amylin, neuropeptide Y (NPY), pancreatic polypeptide, substance P, oxyntomodulin, and somatostatin.

The term “dysregulation,” as used herein in connection with an endocrine factor, means and includes abnormality or impairment in the synthesis, production and/or transmission of an endocrine factor and, hence, abnormality or impairment in biological processes modulated by the endocrine factor.

The term “compound”, as used herein, means and includes any composition of matter comprising two or more chemical elements. According to the invention, in some instances, the terms “compound” and “ligand” are synonymous and used interchangeably herein.

According to the invention, the term “compound” means and includes, without limitation, the natural compounds and ligands (referred to herein as “receptor activating compounds” and “receptor antagonizing compounds” and/or ligands) described in priority U.S. application Ser. No. 18/430,796, Ser. No. 18/980,129, and Ser. No. 19/070,810.

The term “compound” thus means and includes, without limitation, the following “natural” compounds and ligands: butyl butyryl lactate, eugenol, farnesol, (−)-carvone, choline, 3,3′,4′,5,6,7,8-heptamethoxyflavone (HMF), benzyl acetate, cinnamaldehyde, spearmint oil, long-chain free fatty acids (e.g., palmitic acid and stearic acid), and medium-chain free fatty acids (e.g., caproic acid (C6:0), caprylic acid (C8:0), capric acid (C10:0), and lauric acid (C12:0)).

The term “natural,” as used herein in connection with a compound or ligand, and a composition of the invention formed therewith, means and includes a compound or ligand that exists or is synthesized in nature without intervention, including, by way of example, a food-based molecule.

The term “natural,” as used herein in connection with a compound or ligand, and a composition of the invention formed therewith, also means and includes a compound or ligand that originally existed or was synthesized in nature without intervention and is subjected to processing without altering the chemical structure of the compound, such as chemical purification and isolation processes and the formulation of compositions from two or more compounds.

The terms “composition”, “formulation”, “olfactory composition” and “olfactory formulation” are used interchangeably herein, mean and include any compound or combination of compounds that can interact with and modulate at least one olfactory receptor and/or ectopic olfactory receptor and/or free fatty acid receptor and/or transient receptor potential ion channel.

The terms “express” and “expression” as used interchangeably herein, mean, and include the production of a protein product from the genetic information contained within a nucleic acid sequence.

The term “upregulation”, as used herein, means, and includes the increased production of a protein product from the genetic information contained within a nucleic acid sequence.

The term “downregulation”, as used herein, means, and includes the decreased production of a protein product from the genetic information contained within a nucleic acid sequence.

The terms “prevent” and “preventing” are used interchangeably herein, and mean and include precluding a disease, physiological disorder, or pathological condition presented by a subject or subject. The term does not require an absolute preclusion of the disease or condition. Rather, this term includes decreasing the chance for disease occurrence and recurrence.

The terms “prevent” and “preventing” also mean and include reducing the frequency or severity of a disease, physiological disorder or pathological condition presented by a subject or subject.

The terms “treat,” “treatment” and “treating” are used interchangeably herein, and mean and include management of a disease, physiological disorder or pathological condition presented by a subject or subject to cure, ameliorate, stabilize, or prevent the disease, physiological disorder or pathological condition. The terms “treat” and “treating” include “active treatment”, i.e., treatment intended to cure or stabilize a disease, physiological disorder or pathological condition, and “causal treatment”, i.e., treatment intended to abate or prevent the cause of the associated disease, physiological disorder or pathological condition.

The terms “treat,” “treatment” and “treating” further include “palliative treatment”, i.e., treatment intended to relieve the symptoms a disease, physiological disorder or pathological condition, “preventative treatment”, i.e., treatment intended to minimize or partially or completely inhibit the development of the associated disease, physiological disorder or pathological condition, and “supportive treatment”, i.e., treatment employed to supplement another treatment modality directed toward curing, ameliorating, stabilizing, or preventing the associated disease, pathological condition, or disorder.

The terms “delivery” and “administration” are used interchangeably herein, and mean and include providing a composition (or formulation), through any method appropriate to deliver the composition (or formulation) to a subject. According to the invention, such administration means includes, without limitation, oral, sublingual, inhalation, intranasal, epidural, intracerebral, transdermal, topical, and injection administration means.

The term “IC₅₀”, as used herein, means, and includes the concentration of an agonist, antagonist, compound and/or ligand, which, after delivery, inhibits or attenuates at least 50% of a biological and/or physiological process.

In some embodiments, the term “IC₅₀” refers to the concentration of a modulator (e.g., an antagonist or compound), which, after delivery, abates, inhibits or attenuates at least 50% of a biological and/or physiological process, e.g., lipogenesis, atherosclerotic plaque formation, maladaptive RAAS activation, and maladaptive remodeling of vascular or myocardial tissue.

The term “EC₅₀”, as used herein, means, and includes the concentration of an agonist, compound and/or ligand, which, after delivery to a subject, induces at least 50% activation of a biological and/or physiological process.

In some embodiments, the term “adapted”, as used in connection with a “compound”, “ligand”, “agonist”, and “antagonist”, means the “compound”, “ligand”, “agonist” and/or “antagonist” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonin receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κB (NK-κB) and an NADPH oxidase (NOX) by virtue of the concentration, i.e., IC₅₀ or EC₅₀ value, of the “compound”, “ligand”, “agonist”, or “antagonist.”

In some embodiments, the term “adapted”, as used in connection with a “composition” and “formulation” also means the “composition” and/or “formulation” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonin receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κB (NK-κB) and an NADPH oxidase (NOX) by virtue the concentration, i.e., IC₅₀ or EC₅₀ value, of a “compound” or “ligand” contained in the “composition” or “formulation.”

In some embodiments, the term “adapted”, as used in connection with a “composition” and “formulation” also means the “composition” and/or “formulation” is capable of inducing or abating one or more biological and/or physiological processes or activities referenced above by virtue of the concentrations, i.e., IC₅₀ or EC₅₀ values, of two (2) or more “compounds”, “ligands”, “agonists”, or “antagonists” in the “composition” or “formulation.”

The term “comprise” and variations of the term, such as “comprising” and “comprises”, means “including, but not limited to” and is not intended to exclude, for example, other compounds, ligands or method steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention.

The present invention is directed to compositions and methods for treating cardiovascular disorders by modulating receptor activity and, thereby, insulin secretion in vivo.

As discussed above, various entities have developed GLP-1 analogs that mimic endogenous GLP-1 alone, and dual GLP-1/GIP analogs that mimic both endogenous GLP-1 and GIP in combination, which, when delivered to a subject, induce secretion of endogenous GLP-1 and GIP, and, thereby, insulin secretion.

As also discussed above, although the GLP-1 analogs and dual GLP-1/GIP analogs can effectively modulate insulin secretion, there are several significant drawbacks and disadvantages associated with administration of GLP-1 analogs and dual GLP-1/GIP analogs to subjects, including, a high risk of hypoglycemia, adverse side effects, and high costs.

As discussed in detail below, Applicant has developed compositions and methods for treating insulin resistance-induced physiological disorders; particularly, cardiovascular disorders, which, when delivered to subjects, effectively and safely modulate endogenous GLP-1 and GIP secretion and, thereby, insulin secretion in vivo, without the undesirable side effects associated with delivery of a GLP-1 analog and/or a dual GLP-1/GIP analog to the subjects.

Although the compositions of the invention are described in connection with the treatment of cardiovascular diseases and disorders and underlying causes thereof, use of the compositions is not limited solely to the treatment of cardiovascular diseases and disorders, and underlying causes thereof. As set forth in Applicant's U.S. application Ser. No. 18/430,796, Ser. No. 18/980,129, Ser. No. 18/892,760, and Ser. No. 19/070,810, which are expressly incorporated by reference herein in their entirety, the compositions can also be employed to effectively treat various additional insulin resistance-induced physiological disorders, such as type 2 diabetes mellitus.

### Pathophysiology of Atherosclerosis

As is well established, atherosclerosis is a chronic immunoinflammatory, fibroproliferative disease in which there is a build-up of plaques inside arteries. The plaques are

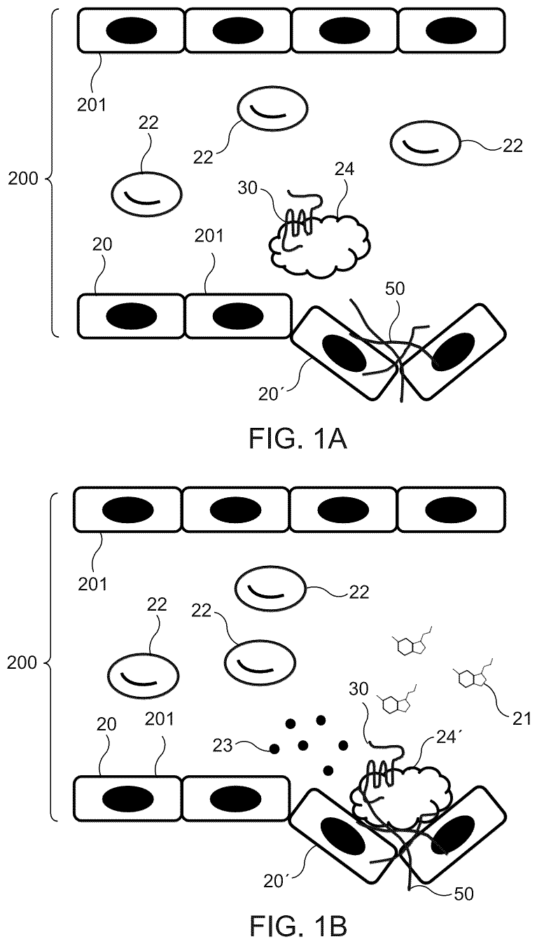

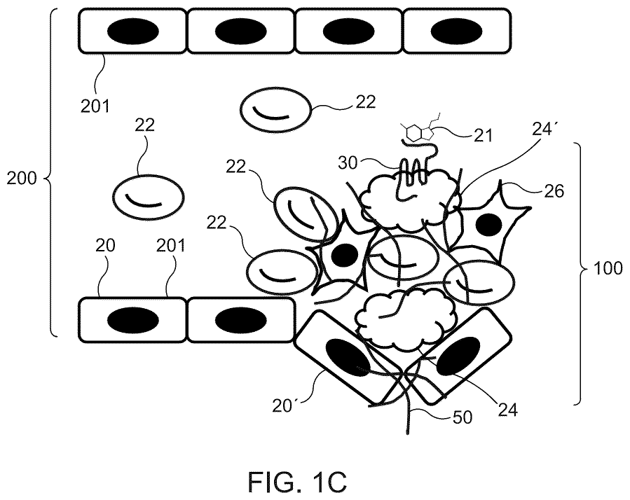

Atherosclerotic plaques are typically formed in response to damage of endothelial cells (denoted “20”) that define the luminal wall (denoted “201”) of an arterial blood vessel (denoted “200”). As illustrated in FIG. 1A, the endothelial cell damage induces in the release of free collagen (denoted “50”) from the damaged endothelial cells (denoted “20′”).

As illustrated in FIGS. 1A and 1B, the free collagen 50 induces the activation of free platelets (denoted “24”) in the blood vessel 200 and adhesion of the activated platelets (denoted “24′”) to the luminal wall 201.

As further illustrated in FIG. 1B, after adhesion of the activated platelets 24′ to the luminal wall 201, the activated platelets 24′ produce and secrete a plurality of cell signaling molecules, which include serotonin (denoted “21”), p-selectin (denoted “23”), and plurality of additional molecules, such as adenosine diphosphate (ADP).

As illustrated in FIG. 1C, after production and secretion of the cell signaling molecules by the activated platelets 24′, the serotonin 21 produced and secreted by the activated platelets 24′ binds to and activates 5-HT₂-serotonin receptors (denoted “30”) (such as 5-HT_{2A}-serotonin receptors) of the activated platelets 24′ and free platelets 24.

As further illustrated in FIG. 1C, the binding of serotonin 21 to the 5-HT₂-serotonin receptors 30 activates the receptors 30 and, thereby, induces activation of proatherogenic cell signaling processes that induce the formation of an atheroma (denoted “100”), which comprises an amalgamation of activated platelets 24′, erythrocytes (or red blood cells) 22, free collagen 50 (and other extracellular matrix (ECM) components and cell debris), macrophages and foam cells (denoted “26”), and a plurality of other atheroma components, such as proatherogenic lipids.

The atheromas 100 can, and in many instances will, induce acute, life-threatening cardiovascular events, such as angina pectoris and cerebral ischemia, and, thereby, severe cardiovascular complications, such as heart failure.

### Correlation Between Diabetes and Atherosclerosis

As discussed in detail below, recent studies reflect that there is also a strong correlation between other insulin-resistance-induced physiological disorders; particularly, type 2 diabetes mellitus, and, hence, the physiological risk factors associated therewith, and the development of atheromas, i.e., atherosclerosis. See, e.g., Goldberg, Ira J., Diabetic Dyslipidemia: Causes and Consequences, Journal of Clinical Endocrinology & Metabolism, v. 86(3), pp. 965-971 (2001); Ormazabal, et al., Association between Insulin Resistance and the Development of Cardiovascular Disease, Cardiovascular Diabetology, v.17, pp. 1-14 (2018); Semenkovich, C. F., Insulin Resistance Atherosclerosis Atherosclerosis, Journal of Clinical Investigation, v. 116(7), pp. 1813-1822 (2006); and O'Leary, et al., Insulin Sensitivity and Atherosclerosis, The Insulin Resistance Atherosclerosis Study (IRAS) Investigators, Circulation, v. 93(10), pp. 1809-17 (1996).

Indeed, as indicated above, the studies reflect that insulin resistance (even at an early stage) induces a proatherogenic lipid phenotype in a pre-diabetic subject. In the initial stages of systemic insulin resistance, serum free fatty acid (FFA) levels are increased due to a decreased suppression of lipolysis in adipocytes. Systemic insulin resistance also abates degradative pathways for apolipoprotein B (apoB) in hepatic tissue, which results in increased LDL production and, hence, increased serum levels of LDL.

The LDLs in the subject's blood then oxidize into oxLDLs that induce the immunogenic release of chemotactic factors from adipocytes that modulate inflammatory responses in adipose tissue, such as MCP-1 and TNFα.

As further indicated above, MCP-1 (and, in some instances and TNFα) initiates the migration of monocytes into visceral adipose tissue (VAT) and promotes their differentiation into mature macrophages. The mature macrophages then secrete large amounts of pro-inflammatory cytokines and, thereby, (i) increase lipolysis, (ii) decrease GLUT4-mediated glucose transport in muscle tissue, and (iii) impair triglyceride biosynthesis and adipocyte storage in VAT, which results in a further increase in circulating serum triglyceride levels and, thereby, ectopic lipid deposition of toxic fatty acid species in extra-adipose tissue.

The mature macrophages also uptake and accumulate circulating oxLDL in a subject's blood and, thereby, transform into foam cells that adhere to the subject's arterial walls and become the foundation of atheromas.

There is, thus, a strong correlation between the physiological risk factors associated with type 2 diabetes mellitus, and atherosclerosis.

As discussed in detail below, the compositions of the invention are adapted to effectively and safely treat atherosclerosis, i.e., inhibit the formation of atheromas, and, in some embodiments, ameliorate and/or stabilize at least one physiological risk factor and/or at least one pathophysiological effect associated with atherosclerosis by, among other physiological processes, (i) antagonizing, i.e. restricting activity of, at least one 5-HT₂-serotonin receptor and, thereby, abating atherosclerotic platelet aggregation and, hence, atheroma formation in vivo, (ii) activating the AMP-activated protein kinase (AMPK) signaling pathway to inhibit LDL synthesis and proinflammatory processes via induced GLP-1 secretion, and (iii) inducing seminal vascular anti-inflammatory processes; particularly, suppression of foam cell formation by induced GLP-1 secretion, thereby, activating GLP-1 receptor signaling induced autophagy, reduction of ACAT1 expression/activity, and inhibition of the PKA/CD36 pathway to inhibit oxLDL uptake and accumulation by macrophages.

As discussed in detail herein, in preferred embodiment, the compositions of the invention comprise at least one natural agonist, i.e., compound or ligand, that is adapted to bind to and activate (and, hence, modulate) at least one receptor, e.g., an ectopic olfactory receptor or free fatty acid receptor, in vivo, and at least one natural antagonist, i.e., receptor antagonizing compound or ligand, that is adapted to bind to and induce abatement of 5-HT₂-serotonin receptor activity in vivo.

### Receptor Activating Compounds and Ligands

According to the invention, suitable natural receptor activating compounds and ligands, which are adapted to modulate insulin secretion via induced receptor activity, include, without limitation, butyl butyryl lactate, eugenol, lauric acid, benzyl acetate, and cinnamaldehyde.

As discussed in detail herein, in a preferred embodiment of the invention, the compositions of the invention comprise at least butyl butyryl lactate and lauric acid.

As set forth in Applicant's priority U.S. application Ser. No. 18/430,796, Ser. No. 18/980,129, and Ser. No. 19/070,810, and discussed further below, butyl butyryl lactate is adapted to bind to activate at least olfactory receptor family 51 subfamily E member 1 (OR51E1), and lauric acid is adapted to bind to activate at least free fatty acid receptor 1 (FFAR1) and free fatty acid receptor 4 (FFAR4).

As set forth in Applicant's priority U.S. application Ser. No. 18/430,796, Ser. No. 18/980,129, and Ser. No. 19/070,810, and discussed further below, when butyl butyryl lactate and lauric acid bind to and activate olfactory receptor OR51E1, free fatty acid receptor FFAR1 and/or free fatty acid receptor 4 (FFAR4), GLP-1 and/or peptide Y-Y (PYY) and/or GIP secretion is induced and, thereby, insulin secretion is modulated in vivo.

### Olfactory Receptor-Mediated and FFAR-Mediated GLP-1, PYY and GIP Secretion

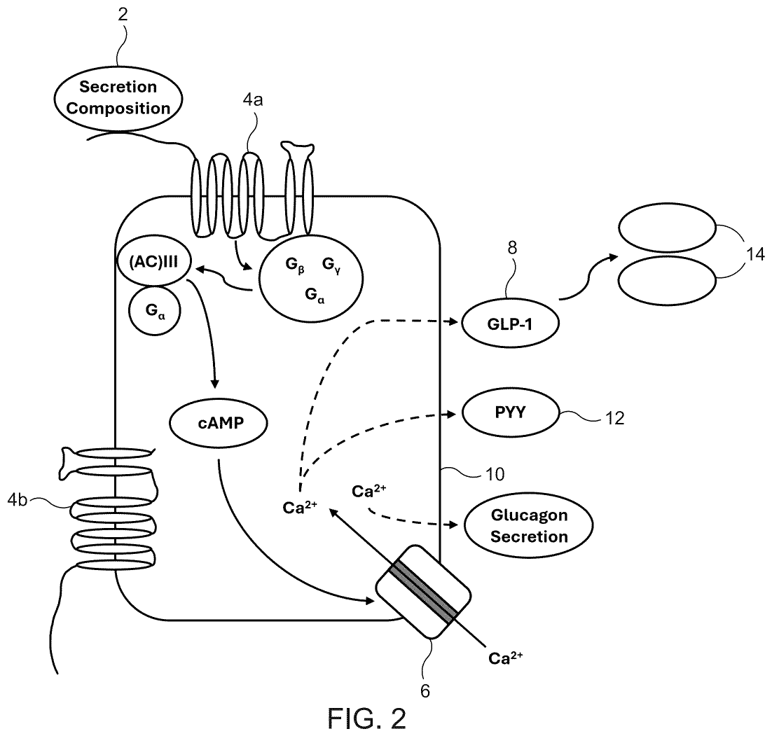

Referring to FIG. 2, according to the invention, when a composition of the invention comprising butyl butyryl lactate (denoted “2”) is delivered to a subject or subject, the composition 2 binds to at least one of the ectopic olfactory receptors (ORs) referenced above (denoted “4a, 4b”) disposed on enteroendocrine cells and pancreatic α-cells, in this instance, enteroendocrine L-cells, as depicted in FIG. 2, wherein a representative enteroendocrine L-cell is denoted “10”.

As illustrated in FIG. 2, the binding of the composition 2 to at least one of the ORs 4a, 4b activates the ORs 4a, 4b, in this instance 4a, by inducing a conformational change in the molecular structure of OR 4a, which activates intracellular G_{α}/G_{β}/G_{γ} subunits of OR 4a, whereby the G_{α}/G_{β}/G_{γ} subunits act as a guanine nucleotide exchange factor, wherein a guanine diphosphate (GDP) is exchanged for a guanine triphosphate (GTP), which binds to the G_{α} subunit of OR 4a.

As further illustrated in FIG. 2, the noted binding of the GTP to the G_{α} subunit induces a dissociation of the G_{α}/G_{β}/G_{γ} subunits of OR 4a into a (i) free G_{α} subunit, which binds to adenyl cyclase (AC) III, and a (ii) G_{β}/G_{γ} complex and, thereby, activates seminal downstream cell signaling processes that induce an increase in intracellular cAMP in the enteroendocrine L-cell 10 and, hence, cells.

The noted increase in intracellular cAMP and a glucose-induced membrane depolarization of the enteroendocrine L-cell 10 (and, hence, cells) opens the voltage-dependent Ca²⁺ (VDC) channels (denoted “6”) of the enteroendocrine L-cells 10, and the resulting Ca²⁺ influx triggers vesicular exocytosis and increases secretion of GLP-1 (denoted “8”) from the enteroendocrine L-cells 10 (and, in some instances, α-cells). In some instances, e.g., when at least one of the activated ORs comprise OR51E1, the resulting Ca²⁺ influx triggers vesicular exocytosis and also increases secretion of PYY (denoted “12”) from the enteroendocrine L-cells 10.

As further illustrated in FIG. 2, the secreted GLP-1 8 binds to and activates GLP-1 receptor proteins on pancreatic β-cells (denoted “14”), which, as indicated above, induces secretion of insulin therefrom.

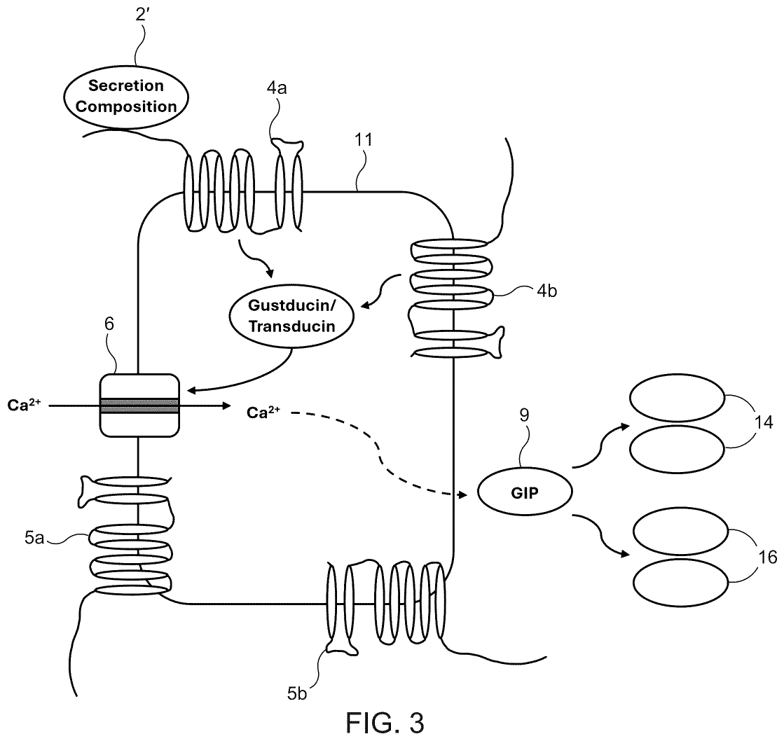

Referring now to FIG. 3, as established by published studies^{1, 2}, when composition of the invention also comprises lauric acid (denoted “composition 2′” binds to at least one of the free fatty acid receptors (denoted “5a, 5b”) disposed on endogenous cells, in this instance, enteroendocrine K-cells, wherein a representative enteroendocrine K-cell is denoted “11”, whereby a gustducin-mediated cell signaling pathway is activated, which induces membrane depolarization of the enteroendocrine K-cell 11 (and, hence, cells) and opens the voltage-dependent Ca²⁺ (VDC) channels (denoted “6”) of the enteroendocrine K-cell(s) 11, wherein the resulting Ca²⁺ influx induces vesicular exocytosis and increased secretion of GIP (denoted “9”) from the enteroendocrine K-cell(s) 11. ¹ See Lee, et al., Therapeutic Potential of Ectopic Olfactory and Taste Receptors, Nature Reviews Drug Discovery, vol. 18, no. 2, pp. 116-138 (2019).² See also Falomir-Lockhart, et al., Fatty Acid Signaling Mechanisms in Neural Cells: Fatty Acid Receptors, Frontiers in Cellular Neuroscience, vol. 13, pg. 162 (2019).

As illustrated in FIG. 3, the secreted GIP 9 binds to and activates GIP receptor proteins on pancreatic β-cells (denoted “14”), which, induces secretion of insulin.

As further illustrated in FIG. 3, the secreted GIP 9 also binds to and activates GIP receptor proteins on endogenous GI cells (denoted “16”), such as islet cells of the pancreas, to promote pancreatic β-cell survival and prevent apoptosis of pancreatic β-cells by activating the cAMP response element-binding (CREB) and Akt/PKB pathways, thus, directly, and indirectly maintaining a stable population of insulin-producing pancreatic β-cells in vivo.

As discussed in detail below, Applicant's analyses also found that an unexpected, unique synergism by and between butyl butyryl lactate and lauric acid will be effectuated when butyl butyryl lactate and lauric acid are combined in preferred compositions of the invention.

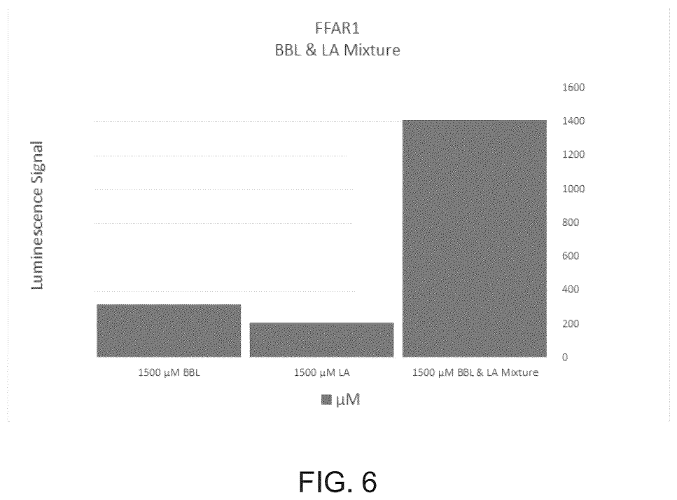

Indeed, as reflected in FIG. 6, the induced FFAR1 activity and, hence, insulinogenic physiological activity resulting therefrom by butyl butyryl lactate and lauric acid, in combination, is unexpectedly, significantly greater than the established physiological activity induced by butyl butyryl lactate and lauric acid when administered separately to a subject at the same concentrations of butyl butyryl lactate and lauric acid in the preferred compositions.

In some embodiments of the invention, the compositions of the invention also comprise one or more natural receptor activating compounds and/or ligands that are specifically adapted to bind to and activate and, hence, modulate transient receptor TRPA1 activity, whereby the following pharmacodynamic activity is induced.

It is believed that, when transient receptor TRPA1 is activated by a natural receptor activating compound or ligand of the invention, the compound/ligand cellular Ca²⁺ is increased, whereby serotonin (5-HT) secretion from endogenous enterochromaffin cells is increased.

The serotonin secreted from the enterochromaffin cells binds to 5-HT receptors of endogenous gastrointestinal cells and, thereby, induces GIP secretion by the endogenous cells.

According to the invention, preferred natural receptor activating compounds and ligands, which are specifically adapted to activate transient receptor TRPA1 in vivo, comprise cinnamaldehyde and allyl isothiocyanate.

According to the invention, the EC₅₀ concentrations of the noted natural receptor activating compounds and ligands contained in a composition of the invention can comprise any EC₅₀ concentration or EC₅₀ concentrations in the range of approximately 1.0 nM to approximately 200.0 mM.

Thus, according to the invention, the EC₅₀ concentrations of natural receptor activating compounds and ligands contained in the compositions of the invention can comprise any of the EC₅₀ concentrations or range of EC₅₀ concentrations set forth in priority U.S. application Ser. No. 18/430,796, Ser. No. 18/980,129, and Ser. No. 19/070,810.

As discussed in detail below, in some embodiments, the compositions of the invention are formed in tablet or capsule form to accommodate oral (or enteric) and sublingual delivery to a subject.

According to the invention, the tablets and capsules comprising the compositions of the invention can comprise any ex vivo mass.

In some embodiments, the ex vivo mass of a composition of the invention is in the range of approximately 600.0 mg to approximately 800.0 mg.

In a preferred embodiment, the ex vivo mass of a natural receptor activating compound or ligands contained in a composition of the invention comprises at least approximately 1.0 μg.

According to the invention, the ex vivo mass of a natural receptor activating compound or ligands contained in a composition of the invention can thus comprise at least approximately 10.0 μg, approximately 15.0 μg, approximately 20.0 μg, approximately 25.0 μg, approximately 30.0 μg, approximately 35.0 μg, approximately 40.0 μg, approximately 45.0 μg, approximately 50.0 μg, approximately 55.0 μg, approximately 60.0 μg, approximately 65.0 μg, approximately 70.0 μg, approximately 75.0 μg, approximately 80.0 μg, approximately 85.0 μg, approximately 90.0 μg, approximately 95.0 μg, approximately 100.0 μg, approximately 1.0 mg, approximately 2.0 mg, approximately 3.0 mg, approximately 4.0 mg, approximately 5.0 mg, approximately 6.0 mg, approximately 7.0 mg, approximately 8.0 mg, approximately 9.0 mg, approximately 10.0 mg, approximately 20.0 mg, approximately 30.0 mg, approximately 40.0 mg, approximately 50.0 mg, approximately 60.0 mg, approximately 70.0 mg, approximately 80.0 mg, approximately 90.0 mg, approximately 100.0 mg, approximately 150.0 mg, approximately 200.0 mg, approximately 250.0 mg, approximately 300.0 mg, approximately 350.0 mg, approximately 400.0 mg, approximately 450.0 mg, approximately 500.0 mg, approximately 600.0 mg, approximately 700.0 mg, approximately 800.0 mg, approximately 900.0 mg, or approximately 1.0 g.

According to the invention, the ex vivo mass of a natural receptor activating compound or ligands contained in a composition of the invention can also comprise in the range of approximately 1.0 μg to approximately 10.0 g.

Thus, according to the invention, the ex vivo mass of a natural receptor activating compound or ligands contained in a composition of the invention can also comprise in the range of approximately 10.0 μg to approximately 50.0 μg, approximately 10.0 μg to approximately 100.0 μg, approximately 10.0 μg to approximately 200.0 μg, approximately 200.0 μg to approximately 400.0 μg, approximately 300.0 μg to approximately 600.0 μg, approximately 400.0 μg to approximately 800.0 μg, approximately 500.0 μg to approximately 1.0 mg, approximately 1.0 mg to approximately 2.0 mg, approximately 1.0 mg to approximately 5.0 mg, approximately 1.0 mg to approximately 10.0 mg, approximately 5.0 mg to approximately 10.0 mg, approximately 5.0 mg to approximately 20.0 mg, approximately 5.0 mg to approximately 30.0 mg, approximately 10.0 mg to approximately 20.0 mg, approximately 10.0 mg to approximately 40.0 mg, approximately 10.0 mg to approximately 50.0 mg, approximately 20.0 mg to approximately 50.0 mg, approximately 20.0 mg to approximately 40.0 mg, approximately 20.0 mg to approximately 60.0 mg, approximately 30.0 mg to approximately 50.0 mg, approximately 30.0 mg to approximately 70.0 mg, approximately 40.0 mg to approximately 80.0 mg, approximately 40.0 mg to approximately 100.0 mg, approximately 100.0 mg to approximately 200.0 mg, approximately 100.0 mg to approximately 300.0 mg, approximately 200.0 mg to approximately 300.0 mg, approximately 200.0 mg to approximately 400.0 mg, approximately 200 mg to approximately 600.0 mg, approximately 100.0 mg to approximately 1.0 g, and approximately 1.0 g to approximately 10.0 g, and/or any ex vivo mass therebetween.

### 5-HT₂-Serotonin Receptor Modulation

In a preferred embodiment of the invention, the compositions of the invention also comprise at least one natural receptor antagonizing compound (or ligand) that is adapted to bind to and antagonize, i.e., restrict activity of, at least one 5-HT₂-serotonin receptor (such as 5-HT_{2A}, 5-HT_{2B} and 5-HT_{2C} serotonin receptors) in vivo (referred to herein as “5-HT₂ serotonin receptor antagonizing compounds”), whereby, as discussed in detail below, atherosclerotic platelet aggregation is attenuated.

As is well established, when a 5-HT₂-serotonin receptor expressed by platelet cells is antagonized, seminal 5-HT₂-serotonin receptor-induced cell signaling processes are abated, including inducement of the Gα_{q} signal transduction pathway. The abated inducement of the Gα_{q} signal transduction pathway prevents that dissociation of the Gα_{q} and β-γ subunits of the 5-HT₂ receptor and, hence, the activation of phospholipase C (PLC) activity by the dissociated Gαq subunit, which subsequently prevents the release of diacylglycerol (DAG) and inositol triphosphate (IP3), and, thereby, inhibits protein kinase C (PKC) activity and intracellular Ca²⁺ release.

The inhibition of protein kinase C (PKC) activity and intracellular Ca²⁺ abates the release of platelet granules, whereby atherosclerotic platelet aggregation induced thereby is prevented and, hence, further treatment of the atherosclerosis is provided.³ ³ See Mohammad-Zadeh, et al., Serotonin: A Review, Journal of Veterinary Pharmacology and Therapeutics, vol. 31.3, pp. 187-199 (2008) and Marcinkowska, et al., Exploring the Antiplatelet Activity of Serotonin 5-HT2A Receptor Antagonists Bearing 6-Fluorobenzo [D] Isoxazol-3-Yl) Propyl) Motif—As Potential Therapeutic Agents in the Prevention of Cardiovascular Diseases Biomedicine & Pharmacotherapy, vol. 145, pg. 112424 (2022).

According to the invention, suitable natural 5-HT₂-serotonin receptor antagonizing compounds comprise, without limitation, 3,3′,4′,5,6,7,8-heptamethoxyflavone (HMF), hesperidin and isoliquiritigenin (ISL).

In a preferred embodiment, the compositions of the invention comprise HMF, which is specifically adapted to bind to and antagonize 5-HT₂-serotonin receptors and, hence, attenuate atherosclerotic platelet aggregation.

HMF will also (i) induce abatement of hepatic fat accumulation and, thereby, reduce hypertriglyceridemia, and (ii) induce an increase in anti-inflammatory interleukin-10 (IL-10) and, thereby, attenuate vascular inflammation and, hence, the development of atherosclerotic plaques promoted thereby.⁴ ⁴ See Nery, et al., Physiological Effects of Tangeretin and Heptamethoxyflavone on Obese C57BL/6J Mice Fed a High-Fat Diet and Analyses of the Metabolites Originating from these Two Polymethoxylated Flavones, Food Science & Nutrition, vol. 9.4, pp. 1997-2009 (2021).

According to the invention, the IC₅₀ concentrations of natural receptor antagonizing compounds and ligands of the invention can comprise any of the IC₅₀ concentrations and ranges of IC₅₀ concentrations set forth in U.S. application Ser. No. 19/070,810 and Ser. No. 18/958,403.

According to the invention, the mass of the noted natural receptor antagonizing compounds and ligands contained in the compositions of the invention can also comprise any of the aforementioned ex vivo masses and mass ranges.

As indicated above, in preferred embodiment, the compositions of the invention comprise at least one natural agonist, i.e., receptor activating compound or ligand, that is adapted to bind to and activate (and, hence, modulate) at least one receptor, e.g., an ectopic olfactory receptor or free fatty acid receptor, in vivo, and at least one natural antagonist, i.e., receptor antagonizing compound or ligand, that is adapted to bind to and induce abatement of 5-HT₂-serotonin receptor activity in vivo.

As also indicated above, in a preferred embodiment, the compositions of the invention comprise at least butyl butyryl lactate, which is adapted to bind to activate at least OR51E1; lauric acid, which is adapted to bind to activate at least FFAR1 and FFAR4; and, optionally, cinnamaldehyde, which is adapted to bind to and activate TRPA1 in vivo (i.e., receptor activating compounds); and HMF, which is adapted to bind to and induce abatement of 5-HT₂-serotonin receptor activity in vivo (i.e., a receptor antagonizing compound).

As further indicated above, the EC₅₀ concentrations of the noted natural receptor activating compounds can comprise any of the EC₅₀ concentrations or range of EC₅₀ concentrations set forth in priority U.S. application Ser. No. 18/430,796, Ser. No. 18/980,129, and Ser. No. 19/070,810, and any of the aforementioned ex vivo masses and mass ranges in the compositions of the invention.

The IC₅₀ concentrations of the noted natural receptor antagonizing compound can comprise any of the aforementioned IC₅₀ concentrations or ranges of IC₅₀ concentrations, and similarly any of the aforementioned ex vivo masses and mass ranges in the compositions of the invention.

By way of example, in one embodiment of the invention, a method for treating atherosclerosis presented by a subject comprises the following steps:
(a) providing a composition comprising (ii) butyl butyryl lactate, the butyl butyryl lactate comprising an EC₅₀ concentration of at least 0.1 μM in the composition, (ii) lauric acid, the lauric acid comprising an EC₅₀ concentration of at least 0.05 μM in the composition, and (iii) 3,3′,4′,5,6,7,8-heptamethoxyflavone (HMF), the HMF comprising an IC₅₀ concentration of at least 0.1 μM in the composition; and
(b) delivering the composition to the subject, wherein secretion of glucagon-like peptide-1 (GLP-1) and gastric inhibitory polypeptide (GIP) is induced, and atherosclerotic platelet aggregation is attenuated.

In another embodiment, a method for treating atherosclerosis presented by a subject comprises the following steps:
(a) providing a composition comprising (ii) butyl butyryl lactate, the butyl butyryl lactate comprising an EC₅₀ concentration of at least 0.1 μM in the composition, (ii) lauric acid, the lauric acid comprising an EC₅₀ concentration of at least 0.05 μM in the composition, (iii) cinnamaldehyde, the cinnamaldehyde comprising an EC₅₀ concentration of at least 0.1 μM in the composition, and (iv) 3,3,4,5,6,7,8-heptamethoxyflavone (HMF), the HMF comprising an IC₅₀ concentration of at least 0.1 μM in the composition; and
(b) delivering the composition to the subject, wherein secretion of glucagon-like peptide-1 (GLP-1) and gastric inhibitory polypeptide (GIP) is induced, and atherosclerotic platelet aggregation is attenuated.

In another embodiment, a method for treating atherosclerosis presented by a subject comprises the following steps:
(a) providing a composition comprising (ii) butyl butyryl lactate, the butyl butyryl lactate comprising an EC₅₀ concentration of at least 0.1 μM in the composition, (ii) eugenol, the eugenol comprising an EC₅₀ concentration of at least 0.1 μM in the composition, (iii) lauric acid, the lauric acid comprising an EC₅₀ concentration of at least 0.05 μM in the composition, (iv) cinnamaldehyde, the cinnamaldehyde comprising an EC₅₀ concentration of at least 0.1 μM in the composition, and (v) 3,3′,4′,5,6,7,8-heptamethoxyflavone (HMF), the HMF comprising an IC₅₀ concentration of at least 0.1 μM in the composition; and
(b) delivering the composition to the subject, wherein secretion of glucagon-like peptide-1 (GLP-1) and gastric inhibitory polypeptide (GIP) is induced, and atherosclerotic platelet aggregation is attenuated.

In yet another embodiment, a method for treating atherosclerosis presented by a subject comprises the following steps:
providing a composition comprising a delivery medium, at least a first receptor activating compound, at least a second receptor activating compound and a receptor antagonizing compound,
the first receptor activating compound comprising butyl butyryl lactate, the butyl butyryl lactate comprising in the range of approximately 3.0% to approximately 5.0% (w/w) of said composition,
the second receptor activating compound comprising lauric acid, the lauric acid comprising in the range of approximately 0.1% to approximately 0.5% (w/w) of said composition,
the receptor antagonizing compound comprising 3,3′,4′,5,6,7,8-heptamethoxyflavone (HMF), the HMF comprising in the range of approximately 15.0% to approximately 17.0% (w/w) of said composition,
the composition adapted to induce activation of at least olfactory receptor family 51 subfamily E member 1 (OR51E1) and free fatty acid receptor 1 (FFAR1) activity in vivo, and induce abatement of 5-HT₂-serotonin receptor activity in vivo; and
delivering the composition to the subject, wherein secretion of glucagon-like peptide-1 (GLP-1) and gastric inhibitory polypeptide (GIP) is induced, and atherosclerotic platelet aggregation is abated.

In some embodiments, the butyl butyryl lactate comprises at least 3.0% (w/w) of said composition.

In some embodiments, the lauric acid comprises at least 0.1% (w/w) of said composition.

In some embodiments, the HMF comprises at least 15.0% (w/w) of the composition.

In some embodiments, the composition further comprises a fourth receptor activating compound comprising cinnamaldehyde.

In some embodiments, the cinnamaldehyde comprises at least 24.0% (w/w) of the composition.

As indicated above, in a preferred embodiment, the compositions of the invention are adapted to induce at least 50% activation of at least olfactory receptor OR51 E1 and free fatty acid receptor FFAR1.

According to the invention, modulating the activity of multiple receptors, e.g., olfactory receptors and/or free fatty acid receptors and/or transient receptor potential ion channels, as described herein, results in elevated endocrine factor levels.

In some embodiments, modulating the activity of multiple receptors, e.g., olfactory receptors and/or free fatty acid receptors and/or transient receptor potential ion channels, as described herein, results in elevated endocrine factor secretion.

In some embodiments, the compositions of the invention further comprise a physiologically suitable (or acceptable) carrier (also referred to herein as a physiologically suitable (or acceptable) excipient, or physiologically suitable (or acceptable) excipient selected based on a chosen route of administration, e.g., oral administration, and standard pharmaceutical practice.

According to the invention, suitable aqueous and non-aqueous carriers that can be employed in the compositions of the invention include water, ethanol, polyols (such as glycerol, glycerin-based water, propylene glycol, polyethylene glycol, and the like), and suitable mixtures thereof; vegetable oils, such as olive oil; buffers, such as neutral buffered saline, phosphate buffered saline and the like; carbohydrates, such as glucose, mannose, sucrose, and dextran, mannitol; proteins; polypeptides, and amino acids, such as glycine; antioxidants; chelating agents such as ethylenediaminetetraacetic acid (EDTA) or glutathione; adjuvants (e.g., aluminum hydroxide); and injectable organic esters, such as ethyl oleate and cyclodextrins.

According to the invention, the compositions of the invention can be manufactured by means of conventional mixing, dissolving, granulating, dragee-making, levigating, emulsifying, encapsulating, entrapping, and lyophilizing processes. The manufactured compositions can take the form of solutions, suspensions, emulsion, tablets, pills, pellets, capsules, capsules containing liquids, powders, sustained-release formulations, suppositories, emulsions, aerosols, sprays, suspensions, and other forms suitable for administration to a subject.

In some embodiments, proper fluidity of a composition is maintained via coating materials, such as lecithin.

According to the invention, the compositions of the invention can be formulated into any known form suitable for parenteral administration, e.g., injection or infusion. Alternatively, as indicated above, the compositions can be formulated for oral administration, nasal or other mucosal tissue administration, or administration as a suppository (e.g., for small molecules). The compositions can also comprise formulation additives, such as suspending agents, preservatives, stabilizers and/or dispersants, and preservation agents.

According to the invention, the compositions can thus be administered to a subject via any suitable method, including, without limitation, oral, sublingual, inhalation, intranasal, epidural, intracerebral, transdermal, topical, and injection administration means.

The compositions of the invention can also be administered to a subject via intraarterial, subcutaneous, intradermal, intratumoral, intranodal, intramedular, intramuscular, intranasally, and intraperitoneal means.

According to the invention, the compositions of the invention can be administered at any of the following dosage ranges: from about 1.0 μg/kg to about 1.0 kg/kg, about 10.0 μg/kg to about 100.0 g/kg, about 10.0 μg/kg to about 25.0 mg/kg, about 100.0 μg/kg to about 50.0 g/kg, about 100.0 μg/kg to about 50.0 mg/kg, about 500.0 μg/kg to about 25.0 g/kg, about 500.0 μg/kg to about 100.0 mg/kg, about 1.0 mg/kg to about 10.0 g/kg, about 1.0 mg/kg to about 50.0 mg/kg, about 5.0 mg/kg to about 5.0 g/kg, about 5.0 mg/kg to about 25.0 mg/kg, about 10.0 mg/kg to about 2.5 g/kg, about 10.0 mg/kg to about 200.0 mg/kg, about 25.0 mg/kg to about 1.5 g/kg, about 25.0 mg/kg to about 750.0 mg/kg, about 50.0 mg/kg to about 1.0 g/kg, about 50.0 mg/kg to about 600.0 mg/kg, about 75.0 mg/kg to about 550.0 mg/kg, about 100.0 mg/kg to about 500.0 mg/kg, about 150.0 mg/kg to about 400.0 mg/kg, and about 200.0 mg/kg to about 350.0 mg/kg.

The compositions of the invention can also be administered at any dosage between the above referenced dosage ranges.

The compositions of the invention can thus also be administered at a dosage of at least about 1.0 μg/kg, at least about 10.0 μg/kg, at least about 100.0 μg/kg, at least about 500.0 μg/kg, at least about 1.0 mg/kg, at least about 5.0 mg/kg, at least about 10.0 mg/kg, at least about 25.0 mg/kg, at least about 50.0 mg/kg, at least about 75.0 mg/kg, at least about 100.0 mg/kg, at least about 150.0 mg/kg, and at least about 200.0 mg/kg.

According to the invention, the compositions of the invention can also be administered at one of the dosage ranges (and/or dosages therebetween) over a prescribed time, by way of example, from about 1.0 μg to about 1.0 kg per day, from about 100.0 μg to about 500.0 g per day, from about 500.0 μg to about 100.0 g per day, from about 1.0 mg to about 20.0 g per day, from about 2.5 mg to about 15.0 g per day, from about 5.0 mg to about 10.0 g per day, from about 10.0 mg to about 5.0 g per day, from about 25.0 mg to about 2.5 g per day, from about 50.0 mg to about 2.0 g per day, from about 100.0 mg to about 1.5 g per day, from about 150.0 mg to about 1.0 g per day, from about 200.0 mg to about 750.0 mg per day, and from about 250.0 mg to about 500.0 mg per day.

According to the invention, the noted dosages and delivery protocols are sufficient to induce sustained (i.e., extended periods) of GLP-1, PYY and/or GIP secretion in vivo.

As discussed in detail above, the compositions of the invention, when delivered to a subject, will effectively and safely treat atherosclerosis, i.e., inhibit the formation of atheromas, and, in some embodiments, ameliorate and/or stabilize at least one physiological risk factor and/or at least one pathophysiological effect associated with atherosclerosis by, among other physiological processes, (i) activating the AMP-activated protein kinase (AMPK) signaling pathway to inhibit LDL synthesis and proinflammatory processes via induced GLP-1 secretion, (ii) inducing seminal vascular anti-inflammatory processes; particularly, suppression of foam cell formation by induced GLP-1 secretion, thereby, activating GLP-1 receptor signaling induced autophagy, reduction of ACAT1 expression/activity, and inhibition of the PKA/CD36 pathway to inhibit oxLDL uptake and accumulation by macrophages, and (iii) antagonizing, i.e. restricting activity of, at least one 5-HT₂-serotonin receptor and, thereby, abating atherosclerotic platelet aggregation and, hence, atheroma formation in vivo.

### EXAMPLE(S)

The following example is provided to enable those skilled in the art to more clearly understand the present invention. The example should not be considered as limiting the scope of the invention, but merely as representative thereof.

### Example I

### Study of In Vitro OR51 E1 and FFAR1 Activity Induced by Butyl Butyryl Lactate and Lauric Acid

The purpose of the following example was to assess the in vitro activity of OR51E1 and FFAR1 induced by butyl butyryl lactate (BBL) and lauric acid (LA), which, as indicated above, would be reflective of downstream GLP-1, GIP, and PYY secretion.

As discussed in detail below, the in vitro activity of BBL and LA was determined by measuring the response of human OR51 E1 and FFAR1 receptors to BBL and LA in a cAMP activity assay.

### cAMP Activity Assay

The cAMP activity assay comprised Hek293T cells transfected with human OR51E1 receptors and HEK293T cells transfected with human FFAR1 receptors.

The HEK293T cells were also transfected with the DNA for the cAMP response element (CRE), which was coupled to a promoter and the gene for firefly luciferase (CRE luciferase). The HEK293T cells were also transfected with DNA for constitutively generated Renilla luciferase.

During the incubation step of the cAMP activity assay, a luciferase substrate (i.e., luciferin compound) was introduced to the transfected HEK293T cells, and firefly luciferase enzymes produced by the HEK293T cells converted luciferin to oxyluciferin and, thereby, produced detectable bioluminescence. The detectable bioluminescence was then measured using a conventional plate reader, which detected and measured the luminescence and provided the readout data for the cAMP activity assay.

### Results

### BBL Induced OR51E1 Activity

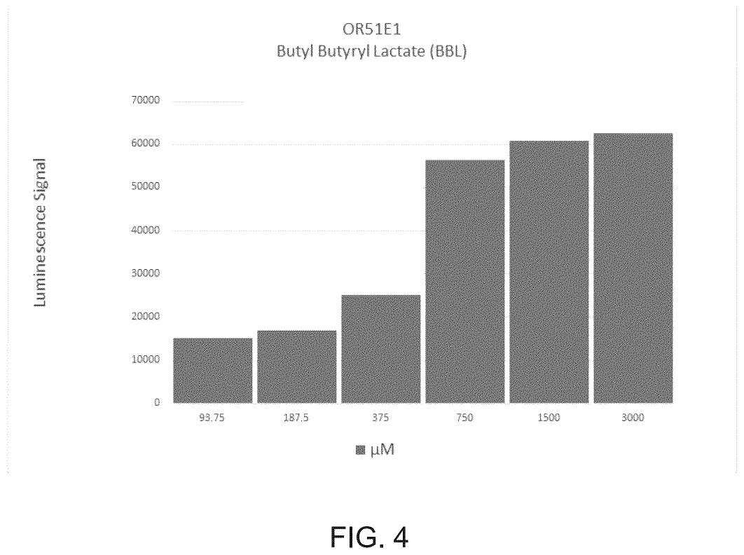

Referring now to FIG. 4, there is shown a bar graph reflecting the induced OR51E1 activity, expressed as luminescence values, by samples of BBL at concentrations of 93.75 μM, 187.5 μM, 375.0 μM, 750.0 μM, 1500.0 μM, and 3000.0 μM.

As reflected by FIG. 4, BBL at concentrations of 93.75 μM and above yielded significant levels of luminescence by the cells and, hence, unexpectedly marked inducement of OR51 E1 activity in vitro.

Although prior studies by prominent researchers, including Saito, et al., Odor Coding by a Mammalian Receptor Repertoire, Science Signaling, vol. 2, no. 60 (2009): ra9-ra9, disclose that BBL can induce OR51 E1 activity at EC₅₀ concentrations ≥1.0 mM, Applicant's study thus establishes that concentrations of BBL substantially lower than 1.0 mM (1000.0 μM), i.e., concentrations of BBL at 93.75 μM, 187.5 μM, 375.0 μM, and 750.0 μM, can and will induce significantly elevated levels OR51E1 activity in vitro.

Applicant's study thus not only confirms induced OR51 E1 activity by BBL, but also unexpectedly reflects that BBL will induce significantly elevated levels OR51E1 activity in vitro at concentrations substantially lower than 1000.0 μM.

### LA Induced FFAR1 Activity

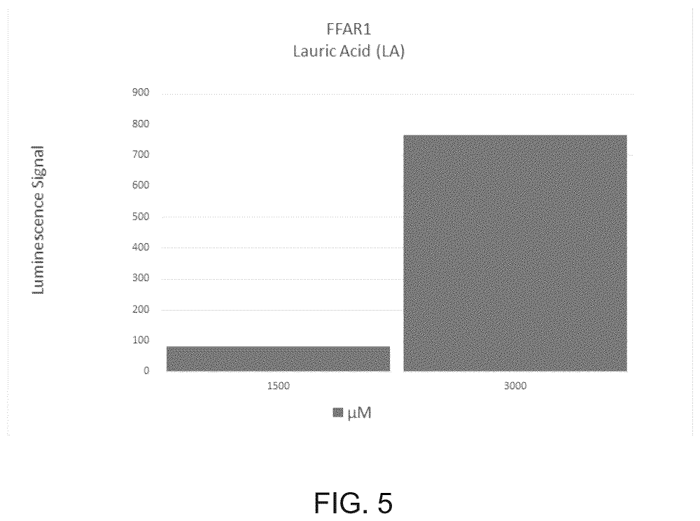

Referring now to FIG. 5, there is shown a bar graph reflecting induced FFAR1 activity, expressed as luminescence values, by samples of LA at concentrations of 1500.0 μM and 3000.0 μM.

As reflected by FIG. 5, LA at concentrations of 3000.0 μM and above yielded significant luminescence by the cells and, hence, significant inducement of FFAR1 activity in vitro.

In view of prior published studies⁵ of induced receptor activity by LA, it was anticipated that significant luminescence produced by the cells and, hence, significant levels of FFAR1 activity, would also have been exhibited by the cells in vitro at concentrations of ≤3000.0 μM. However, as discussed below, due to Applicant's test protocol; specifically, the use of a water-based buffer, a readable reliable luminescence signal was difficult to acquire. ⁵ See Lee, et al. and Falomir-Lockhart, et al., supra.

As is well established, LA is virtually insoluble in water. Indeed, LA's solubility in water at 25° C. is 4.81 mg/L.

It is Applicant's position, which Applicant respectfully submits is in accord with established assay protocol(s), insolubility in a water-based buffer significantly limits the ability of the FFAR1 receptors of the cells to bind to the LA in the water-based buffer, which results in lower levels of luminescence produced by the cells. Difficulty detecting the luminescence produced by the cells at such low levels exasperates luminescence signal interference, such as background luminescence associated with non-specific binding and the laboratory environment.

It is further hypothesized by Applicant that it is possible that the basal or baseline behavior of the FFAR1 transfected cells in vitro also contributed to the difficulty acquiring a readable reliable luminescence signal at LA concentrations of ≤3000.0 μM.

Notwithstanding the low, and Applicant submits, inaccurate, luminescence signals at LA concentrations of ≤3000.0 μM reflected in FIG. 5, Applicant's study confirms, and FIG. 5 depicts, that LA can and will induce significant levels of FFAR1 activity, which is consistent with published studies.

Applicant further respectfully submits that it is highly likely that significant levels of FFAR1 activity would be exhibited by the cells at LA concentrations ≤3000.0 μM if LA was sufficiently solubilized in a specialized assay buffer to facilitate improved binding of the LA to the FFAR1 receptors of the cells.

### BBL & LA Mixture Induced FFAR1 Activity

Referring now to FIG. 6, there is shown a bar graph reflecting the induced FFAR1 activity, expressed as luminescence values, by 1500.0 μM samples of BBL and LA, and a mixture of the BBL and LA.

Although BBL is not generally known as a FFAR1 ligand, as reflected in FIG. 6, when LA (a known FFAR1 ligand) is combined with LA, the BBL and LA mixture induces a level of luminescence that is markedly greater, i.e., approximately seven (7)-fold greater, than the luminescence induced by either BBL or LA alone, thus, evidencing unexpectedly enhanced, synergistic activity by and between BBL and LA, which results in significant inducement of FFAR1 activity and, thereby, insulinogenic physiological activity.

Although the unexpectedly enhanced, synergistic activity by and between BBL and LA and, hence, significant inducement of FFAR1 activity resulting thereby was effectuated by a composition that included a 1:1 ratio of BBL and LA, it is respectfully submitted (and has been shown in preliminary unpublished studies) that the unexpectedly enhanced, synergistic activity by and between the BBL and LA could have and, hence, will also be effectuated at ratios of BBL to LA that fall within the weight percentages of BBL and LA in the compositions of the invention; particularly, compositions comprising in the range of approximately 1.0 to 3.0% (w/w) of BBL and in the range of approximately 0.05 to 0.2% (w/w) LA.

As also reflected by FIG. 6, although, as indicated above, BBL is not generally known as a FFAR1 ligand, BBL at a concentration of 1500.0 μM induced significant levels of luminescence by the cells and, hence, reflected an unexpectedly marked inducement of FFAR1 activity in vitro.

Contrary to published studies, it is thus highly likely that FFAR1 possesses functional binding affinity for BBL.

Applicant's study thus not only establishes induced FFAR1 activity by BBL alone, but also unexpectedly reflects enhanced, synergistic activity by and between BBL and LA at weight percentages in compositions of the invention, which results in significant inducement of FFAR1 activity.

As will readily be appreciated by one having ordinary skill in the art, the present invention provides numerous advantages compared to prior art formulations and methods for treating insulin resistance-induced physiological disorders. Among the advantages are the following:
The provision of natural compounds and ligands, and compositions comprising same, for treating physiological disorders; particularly, insulin resistance-induced physiological disorders, such as type 2 diabetes mellitus, and cardiovascular disorders, which substantially reduce or overcome the drawbacks and disadvantages associated with administration (or delivery) of GLP-1 analogs and dual GLP-1/GIP analogs to subjects presenting with a physiological disorder.
The provision of natural compounds and ligands, and compositions comprising same, which, when administered to subjects presenting with a physiological disorder, effectively treat the physiological disorder by modulating ectopic olfactory receptor activity;
The provision of natural compounds and ligands, and compositions comprising same, which effectuate olfactory receptor (OR)-mediated secretion of endogenous GLP-1 and PYY in subjects presenting with a physiological disorder, without the undesirable side effects associated with delivery of a GLP-1 analog and/or a dual GLP-1/GIP analog to subjects;
The provision of natural compounds and ligands, and compositions comprising same, which, when administered to subjects presenting with a cardiovascular disorder, effectively and safely modulate the subjects'insulin secretion in vivo, whereby the cardiovascular disorder is effectively treated and at least one risk factor associated with the cardiovascular disorder is ameliorated with minimal, if any, adverse side effects;
The provision of natural compounds and ligands, and compositions comprising same, which, when administered to subjects presenting with a cardiovascular disorder, effectively and safely modulate the subjects'insulin secretion and abates 5-HT₂-serotonin receptor activity in vivo, whereby the cardiovascular disorder is effectively treated and at least one risk factor associated with the cardiovascular disorder is ameliorated with minimal, if any, adverse side effects; and
The provision of natural compounds and ligands, and compositions comprising same, which, when administered to subjects, effectively and safely modulate the subjects'systemic insulin secretion and abates 5-HT₂-serotonin receptor activity in vivo, and can be administered to the subjects via oral, sublingual, inhalation, intranasal, epidural, intracerebral, transdermal, topical, and injection administration means.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for treating atherosclerosis presented by a subject, comprising the steps of:
providing a composition comprising a delivery medium, at least a first receptor activating compound, at least a second receptor activating compound and a receptor antagonizing compound,
said first receptor activating compound comprising butyl butyryl lactate, said butyl butyryl lactate comprising in the range of approximately 3.0% to approximately 5.0% (w/w) of said composition,
said second receptor activating compound comprising lauric acid, said lauric acid comprising in the range of approximately 0.1% to approximately 0.5% (w/w) of said composition,
said receptor antagonizing compound comprising 3,3′,4′,5,6,7,8-heptamethoxyflavone (HMF), said HMF comprising in the range of approximately 15.0% to approximately 17.0% (w/w) of said composition,
said composition adapted to induce activation of at least olfactory receptor family 51 subfamily E member 1 (OR51E1) and free fatty acid receptor 1 (FFAR1) activity in vivo, and induce abatement of 5-HT₂-serotonin receptor activity in vivo; and
delivering said composition to said subject, wherein secretion of glucagon-like peptide-1 (GLP-1) and gastric inhibitory polypeptide (GIP) is induced, and atherosclerotic platelet aggregation is abated.

2. The method of claim 1, wherein said butyl butyryl lactate comprises at least 3.0% (w/w) of said composition.

3. The method of claim 1, wherein said lauric acid comprises at least 0.1% (w/w) of said composition.

4. The method of claim 1, wherein said HMF comprises at least 15.0% (w/w) of said composition.

5. The method of claim 1, wherein said composition further comprises a third receptor activating compound comprising eugenol, said eugenol comprising at least 3.0% (w/w) of said composition.

6. The method of claim 1, wherein said composition further comprises a fourth receptor activating compound comprising cinnamaldehyde, said cinnamaldehyde comprising at least 24.0% (w/w) of said composition.

7. The method of claim 1, wherein said composition further comprises a fifth receptor activating compound comprising benzyl acetate, said benzyl acetate comprising at least 0.5% (w/w) of said composition.

8. The method of claim 1, wherein said composition further comprises a sixth receptor activating compound comprising spearmint oil, said spearmint oil comprises at least 7.0% (w/w) of said composition.

9. The method of claim 1, wherein said delivery medium comprises sunflower seed oil.
